(19) United States
(12) Patent Application Publication
(10) Pub. No.: US 2010/0112684 A1
(43) Pub. Date: May 6, 2010

(54) CELL CULTURE DISH FOR THE EMBRYOID BODY FORMATION FROM EMBRYONIC STEM CELLS

(75) Inventors: Min Young Lee, Gwangju (KR); Ho Jae Han, Gwangju (KR)

Correspondence Address: CANTOR COLBURN, LLP, 20 Church Street, 22nd Floor, Hartford, CT, 06103, US

(73) Assignee: INDUSTRY FOUNDATION OF CHONNAM NATIONAL UNIVERSITY, Gwangju (KR)

(21) Appl. No.: 12/594,982

(22) PCT Filed: Apr. 8, 2008
(86) PCT No.: PCT/KR2008/001987

§ 371 Date: Oct. 26, 2009

(30) Foreign Application Priority Data
Apr. 9, 2007 (KR) 10-2007-0034482

(51) Int. Cl. C12M 1/22

(52) U.S. Cl. 435/305.1

(57) ABSTRACT

The present invention relates to a cell culture dish for the erabryoid body formation from embryonic stem cells, which facilitates efficient and stable differentiation of embryonic stem cells by forming embryoid body in grooves on the bottom of a support adhered on the back of the lid for the culture dish by hanging drop culture.

### TECHNICAL FIELD

The present invention relates to a cell culture dish for the embryoid body formation in embryonic stem cell differentiation process, more precisely a cell culture dish for the embryoid body formation from embryonic stem cells, which facilitates efficient and stable differentiation of embryonic stem cells by forming embryoid body in grooves on the bottom of a support adhered on the back of the lid for the culture dish by hanging drop culture.

### BACKGROUND ART

For biological studies to disclose characteristics of embryonic stem cells and for cell therapy, differentiation of embryonic stem cells into target cells having specific function is induced. The most representative and widely known method for the differentiation is the one composed of the following steps: embryonic stem cells are cultured and recovered to form embryoid body by hanging drop culture; the embryoid body is suspension-cultured and the floated embryoid body is recovered; the embryoid body is attached-cultured in the medium for differentiation.

FIG. 1 is a concept map illustrating the conventional method to form an embryoid body by hanging drop culture.

The embryoid body formation is performed as follows. As shown in FIG. 1(a), the recovered cells were counted and approximately 300˜500 cells were distributed on the lid (110) of the conventional cell culture dish (100) as drops by using a pipette, resulting in the preparation of reverse attached culture, which is hanging drop culture. As shown in FIG. 1(b), the hanging cells are gathered around the tip of a drop by gravity, so that cell mass in the form of embryo is formed, which is called embryoid body.

The formed embryoid body is transferred in suspension culture medium (120) by any random method, followed by suspension culture. During the process of transferring the embryoid body, a tool such as pipette is generally used. At this time, the embryoid body can be damaged by the pressure given by the pipette and as the number of cells increase, more time is required. Besides, during the process of transferring, contamination by microorganism might be a serious problem. In the differentiation of embryonic stem cells, each stage is sophisticated and requires concentration. And, it is very important in the differentiation of embryonic stem cells to form a healthy embryoid body. The differentiation is induced by several steps starting from the culture of undifferentiated embryonic stem cells and each step takes a long time. So, if cells are contaminated in any of those steps by a microorganism, it is very difficult to control such contamination and thus the differentiation processes have to be started all over again, causing severe economic and time loss.

### DISCLOSURE

### Technical Problem

It is an object of the present invention, to overcome the above problem, to provide a cell culture dish favoring the enhancement of differentiation efficiency of embryonic stem cells, which is free from contamination by a microorganism and physical damage of embryoid body caused during the transferring of the embryoid body to the suspension culture medium.

It is another object of the present invention to provide a cell culture dish capable of full supply of nutrition to embryoid body and jumping over suspension culture stage directly to attachment culture.

### Technical Solution

To achieve the above objects, the cell culture dish for the embryoid body formation from embryonic stem cells of the present invention is composed of a Petri-dish providing a space which is deep enough; a lid for the culture dish for opening and closing the Petri-dish; Supports placed at the bottom of a lid (20) and multiple supports where regular grooves are formed at the tip of the bottom of the lid.

The cell culture dish herein can be prepared by one of polycarbonate, polyethylene, polypropylene or glass.

The groove herein can have embossed or depressed protrusions.

The groove is in the shape of circular cone or the screw thread can be additionally formed inside of the groove.

The groove can additionally include a surface-treated layer prepared by plasma irradiation or gamma-ray irradiation to improve adhesive power.

### Advantageous Effect

As explained hereinbefore, the cell culture dish for the embryoid body formation from embryonic stem cells of the present invention favors inhibition of physical damage of the embryoid body caused during the differentiation of embryonic stem cells and minimizes contamination by a microorganism, so that it brings the effect of reducing time and labor for suspension culture.

By forming embryoid body in an appropriate size for the induction of differentiation by hanging drop culture, suspension culture stage can be eliminated or shortened, so that time and economic loss that might be caused during the suspension culture can be prevented.

### DESCRIPTION OF DRAWINGS

The above and other objects, features and advantages of the present invention will become apparent from the following description of preferred embodiments given in conjunction with the accompanying drawings, in which:

FIG. 1 is a cross-section of an example illustrating the conventional cell culture dish for the embryoid body formation.

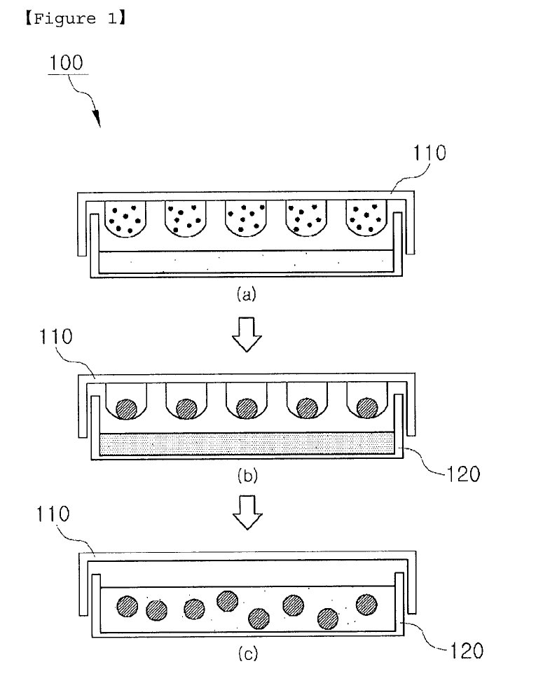

FIG. 2 is an oblique view of the cell culture dish for the embryoid body formation from embryonic stem cells of the present invention.

FIG. 3 is a cross-section of example 1 illustrating the cell culture dish for the embryoid body formation of the present invention.

FIG. 4 is a cross-section of example 2 illustrating the cell culture dish for the embryoid body formation of the present invention.

FIG. 5 is an enlarge section of supports formed on the lid of the cell culture dish of the present invention.

### DETAILED DESCRIPTION OF MAIN ELEMENTS

10: cell culture dish

20: lid

30: support

50: Petri-dish

40: groove

### BEST MODE

Hereinafter, the cell culture dish for the embryoid body formation from embryonic stem cells of the present invention is described in more detail with attached figures.

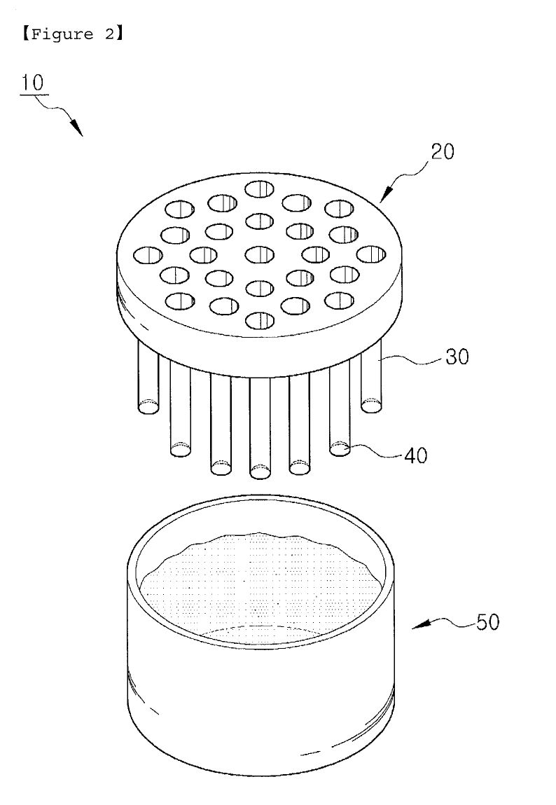

FIG. 2 is an oblique view of the cell culture dish for the embryoid body formation from embryonic stem cells of the present invention.

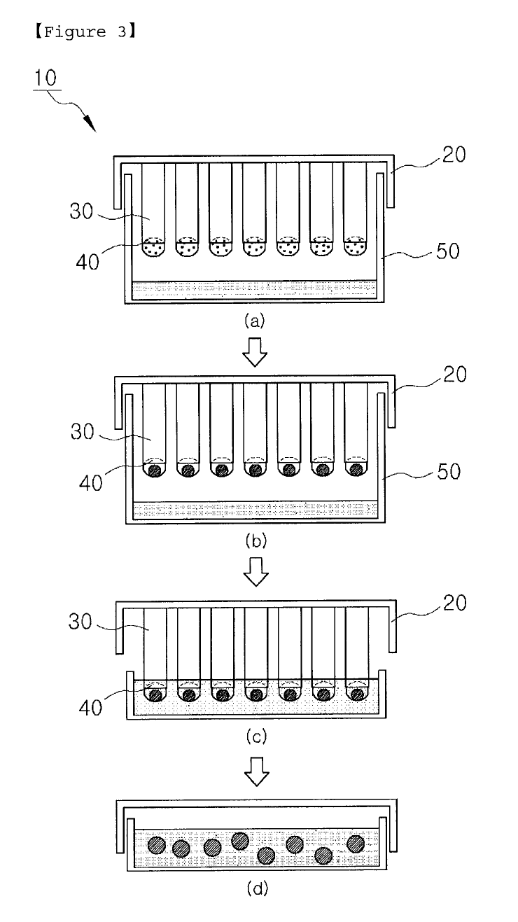

FIG. 3 is a cross-section of example 1 illustrating the cell culture dish for the embryoid body formation of the present invention.

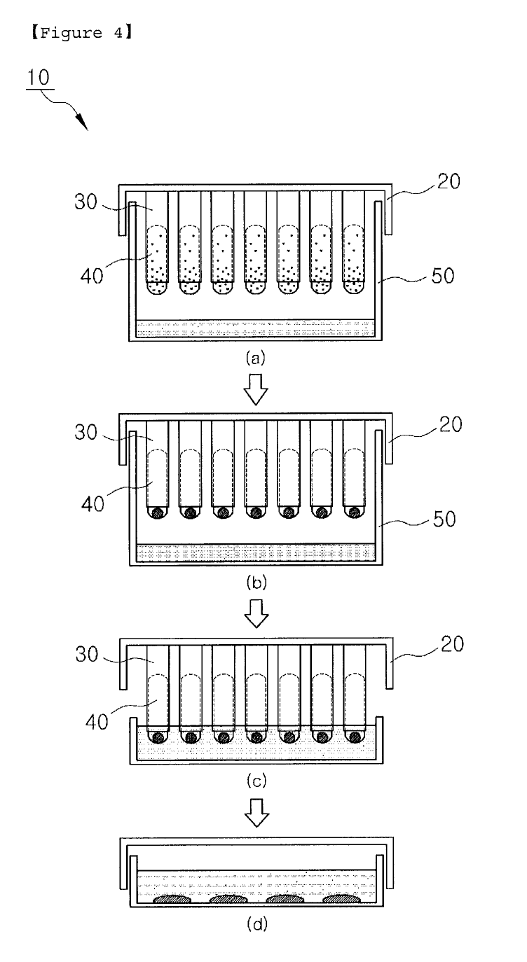

FIG. 4 is a cross-section of example 2 illustrating the cell culture dish for the embryoid body formation of the present invention.

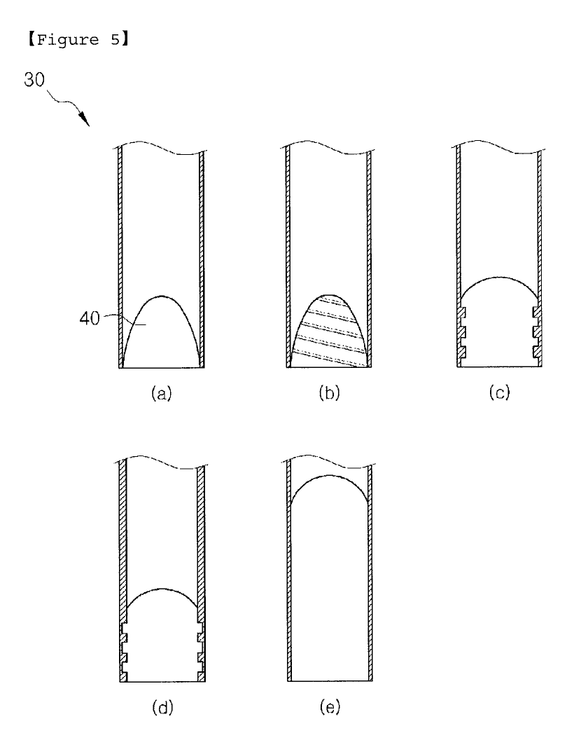

FIG. 5 is an enlarge section of supports formed on the lid of the cell culture dish of the present invention.

As shown in FIG. 2, the cell culture dish for the embryoid body formation from embryonic stem cells of the present invention is composed of a Petri-dish (50) providing a space which is deep enough; a lid (20) for the culture dish for opening and closing the Petri-dish (50); and multiple supports (30) where regular grooves (40) are formed at the tip of the bottom of the lid (20).

The cell culture dish (10) is prepared by one of the materials suitable for cell culture such as polycarbonate, polypropylene, polyethylene or their copolymer or glass, but not always limited thereto. And, a transparent material is preferred, but a colored material can also be used. It is also preferred to have even surface and the shape of round or square, but not always limited thereto. This dish can be specifically treated by inserting a specific pattern on the substrate and modified in its shape for the purpose of the use or cell specificity.

Multiple supports (30) in the shape of circular cylinder are formed at regular length on the bottom of the lid (20). The supports (30) are not necessarily in the shape of circular cylinder, and can be in the shape of square or polygon.

The grooves (40) are formed in a regular depth from the tip of the bottom of the supports (30) to the upper direction. As shown in FIG. 5, the grooves (40) can be formed in different depths and the inside of the groove (40) has embossed or depressed protrusions or screw thread to improve cell adhesiveness. The groove (40) can be in the shape of circular cone.

The cell culture dish (10) can additionally include a cell growth layer to support the growth of cells after being fixed or floated. This cell growth layer can be a thin film composed of any material that is capable of supporting cell growth and not-toxic to cells, which is exemplified by polylysine, collagen, gelatin, laminin, matrigel and cell matrix component.

The groove (40) can be treated by plasma gas or gamma-ray irradiation to improve cell adhesiveness according to the characteristics of cell culture and the purpose of the use, but not always limited thereto. The surface-treated layer is preferably transparent, but can also be colored.

The Petri-dish (50) needs to be fit to the lid (20), so that the lid (20) can be covered with the Petri-dish (50) for tight holding. The Petri-dish (50) is prepared to have enough depth, so that the supports (30) cannot touch the inside bottom. In the inside, culture medium or PBS (Phosphate Buppered Saline) is loaded to prevent the embryoid body from being dried during the embryoid body formation.

FIG. 3 illustrates the embryoid body formation using the cell culture dish for the embryoid body formation shown in FIG. 2.

As shown in FIG. 3(a), when hanging drop culture is performed in the groove (40) of the support (30), embryoid body is formed as shown in FIG. 3(b). As shown in FIG. 3(c), the lid (20) is dipped in the culture dish containing suspension culture medium, then the embryoid body is moved to the suspension culture medium as shown in FIG. 3(d). The embryoid body can be transferred without being damaged by simply one-time dipping. And also, multiple embryoid bodies can be moved at a time, so that time and labor are saved and clean process is achieved.

FIG. 4 illustrates the embryoid body formation using the cell culture dish for the embryoid body formation having the grooves (40) of FIG. 5(e).

As shown in FIG. 4(a), hanging drop culture is performed in grooves (40) formed deep and to the upper direction to provide enough medium to embryoid body. When embryoid body is formed large enough, suspension culture stage can be omitted and attachment culture follows in the culture dish with the cell growth layer formed thereon. At this time, it is also possible to move embryoid body to attachment culture medium without damaging by simply one-time dipping.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### INDUSTRIAL APPLICABILITY

The cell culture dish for the embryoid body formation from embryonic stem cells of the present invention favors inhibition of physical damage of embryoid body caused during the differentiation of embryonic stem cells and minimizes the contamination by a microorganism, saves time and effort for transferring embryoid body for suspension culture or omits or shortens suspension culture stage, so that this dish has advantage of economic efficiency by saving time and labor for suspension culture.

1. A cell culture dish for the embryoid body formation from embryonic stem cells, which is characteristically composed of a Petri-dish (50) providing a space which is deep enough; a lid (20) for the culture dish for opening and closing the Petri-dish (50); Supports is placed at the bottom of a lid (20) and multiple supports (30) where regular grooves (40) are formed at the tip of the bottom of the lid (20).

2. The cell culture dish for the embryoid body formation from embryonic stem cells according to claim 1, wherein the culture dish (10) is prepared by one of polycarbonate, polyethylene, polypropylene or glass.

3. The cell culture dish for the embryoid body formation from embryonic stem cells according to claim 1, wherein the groove (40) has embossed or depressed protrusions.

4. The cell culture dish for the embryoid body formation from embryonic stem cells according to claim 1, wherein the groove (40) is in the shape of circular cone or the screw thread can be additionally formed inside of the groove.

5. The cell culture dish for the embryoid body formation from embryonic stem cells according to claim 1, wherein the groove (40) additionally includes a surface-treated layer prepared by plasma gas or gamma-ray irradiation to improve adhesive power.

6. The cell culture dish for the embryoid body formation from embryonic stem cells according to claim 2, wherein the groove (40) has embossed or depressed protrusions.

7. The cell culture dish for the embryoid body formation from embryonic stem cells according to claim 2, wherein the groove (40) is in the shape of circular cone or the screw thread can be additionally formed inside of the groove.

8. The cell culture dish for the embryoid body formation from embryonic stem cells according to claim 2, wherein the groove (40) additionally includes a surface-treated layer prepared by plasma gas or gamma-ray irradiation to improve adhesive power.
